(19) United States
(12) Patent Application Publication
(10) Pub. No.: US 2007/0173478 A1
(43) Pub. Date: Jul. 26, 2007

(54) Compositions for the enhanced treatment of depression

(75) Inventor: Andrew Peter Worsley, Kent (GB)

Correspondence Address: STITES & HARBISON PLLC, 1199 NORTH FAIRFAX STREET, SUITE 900, ALEXANDRIA, VA, 22314, US

(73) Assignee: WWK TRUST, Beckenham, Kent (GB)

(21) Appl. No.: 10/560,348

(22) PCT Filed: Jun. 11, 2004
(86) PCT No.: PCT/GB04/02493

§ 371 Date: Mar. 14, 2007

(30) Foreign Application Priority Data
Jun. 12, 2003 (GB) 0313630.6

(51) Int. Cl. A61K 31/714; A61K 31/405; A61K 31/343; A61K 31/137; A61K 31/445

(52) U.S. Cl. 514/52; 514/317; 514/419; 514/469; 514/649

(57) ABSTRACT

For treatment of endogenous depression, there is taken in combination an antidepressant, particularly an SSRI or SNRA, and a precursor or inducer of a neurotransmitter, e.g. L-phenylalanine, tyramine or L-tryptophan. Optionally, the patient also takes vitamin B₁₂.

### TECHNICAL FIELD

The present invention relates to the use of a combined medicament in the enhanced treatment of various forms of depression, particularly forms of endogenous depression. The invention also relates to the preparation of medicaments for such treatments.

### BACKGROUND OF THE INVENTION AND PRIOR ART

Depression is a psychiatric condition resulting from a disorder of mood. Depression has been recognised as a major disease for centuries. In addition to disorder of mood, patients are at risk of self harm, or even suicide attempts, either successful or unsuccessful.

Depression is thought to result from failure of normal neurotransmitter function where there is failure to produce sufficient neurotransmitter. This often arises as a result of neurotransmitter imbalance. Depression may in part arise from altered efficiency of receptor signalling or from a relative deficiency of neurotransmitter.

Detectable depression occurs in approximately 10% of the general population. Some 20% of depressive patients show moderate to severe symptoms, the severity of which is generally thought to be linked to the duration of depression and the level of control using antidepressants.

Depression may be mild, for example taking the form of a mild mood change. Moderate to severe symptoms of depression can result in self harm or even progress to psychosis.

Previous treatments for depression have included tricyclic antidepressants on their own, monamine oxidase inhibitors (MAOI) and selective serotonin re-uptake inhibitors (SSRI). The amino acid L-tryptophan is also effective, but none of the other amino acids have previously shown clinical benefit, either in combination or on their own.

WO 96/11009 discloses treatment of multiple sclerosis and WO 98/01157 discloses the treatment of peripheral neuropathies by some of the combinations of components employed in the present invention.

Vitamin B₁₂ has been proposed for the treatment of B₁₂-deficiency associated neuropathy.

### DISCLOSURE OF THE INVENTION

The present inventor has surprisingly found that a combination of a tricyclic antidepressant, a monamine oxidase inhibitor (MAOI) or a selective serotonin re-uptake inhibitor (SSRI) with an inducer or a precursor of a neurotransmitter can enhance effectiveness in the treatment of depression. While the present invention is believed applicable to any and all depressive illnesses associated with all psychiatric conditions and exogenous and endogenous depression, it is particularly thought effective for endogenous depression, and in particular chronic endogenous depression and acute non-psychotic endogenous depression. In addition, the combination of an SSRI with L-tryptophan is believed to be surprisingly effective; this combination has the advantage of use of a low dose of L-tryptophan. The components of the medicament of this invention may be presented as a combined preparation for simultaneous, separate or sequential use in the treatment of various forms of depression. It has also been observed that a parallel or simultaneous administration of vitamin B₁₂ treatment, for example orally or by injection, may enhance the therapeutic effect of this combination.

It has also been found that combinations (i) vitamin B₁₂ with an inducer or a precursor of a neurotransmitter and (ii) vitamin B₁₂ with an antidepressant, are effective in treatment of depression.

Accordingly, in a first aspect the present invention provides the use of any one of the following components or combinations of components:
C,
A and B,
A and C or C′,
B and C or C′,
A, B and C or C′,
   wherein
A is an antidepressant,
B is vitamin B₁₂, and
C is a precursor or inducer of a neurotransmitter (other than L-tryptophan),
C′ is L-tryptophan,
   in the manufacture of a medicament for the treatment of at least one form of depression.

In another aspect the invention provides a method of making a medicament for the treatment of a patient suffering from depression, comprising admixing any one of the following components:
C,
A and B,
A and C or C′,
B and C or C′,
A, B and C or C′,
   wherein
A is an antidepressant,
B is vitamin B₁₂, and
C is a precursor or inducer of a neurotransmitter (other than L-tryptophan),
C′ is L-tryptophan,
   with at least one pharmaceutically acceptable component or vehicle to prepare a medicament suitable for administration to a patient.

In yet another aspect the invention provides a method of treatment of a patient suffering from a form of depression, comprising administering to the patient any one of the following combinations of components:
I. A, B and C or C′
II. A and B
III. B and C or C′
IV. A and C or C′
   wherein
A is an antidepressant,
B is vitamin B₁₂, and
C is a precursor or inducer of a neurotransmitter (other than L-tryptophan),
C′ is L-tryptophan,
   said components being administered simultaneously or separately, in amounts which in combination have the effect of ameliorating the depressive condition.

In a further aspect the invention provides a pharmaceutical composition containing as the only pharmaceutically active components or including as the components any combination as set out above.

Treatment may be simultaneous or separate including sequential administration of the components.

In the medicaments of the invention, there may be included at least one pharmaceutically acceptable component or vehicle such as an incipient, carrier, buffer, stabiliser or other material, as discussed below.

Also provided is a kit or pack containing components A and B, or A and C or C′, or A and B and C or C′, or B and C or C′, wherein A, B, C and C′ are as defined above, the components being formulated for simultaneous, separate or sequential delivery in the treatment of depression. Particularly components A and C or C′ may be combined, and component B separate.

The depression with which the present invention is concerned may be characterised by its chronic nature or its acute non-psychotic nature as a result of the neurotransmitter disturbance.

Antidepressants useful in this invention fall into the following known classes:

tricyclics

tetracyclics

selective serotonin re-uptake inhibitors (SSRI) serotonin and noradrelanine re-uptake inhibitors (SNRI)

monoamine oxidase inhibitors, A-type (MA01 A-type, also known as MAO-A inhibitors).

Preferred are antidepressants exhibiting SSRI or SNRI activity, for example

fluoxetine (SSRI)

lofepramine (principally tricyclic but has some SSRI and SNRI activity)

citalapram (SSRI).

Other suitable antidepressants include mianserin, trimipramine, imipramine, clomipramine, amitriptyline, protriptyline, nortriptyline, fluvoxamine, maprotiline, sertaline, venlaflaxine, pargyline, triazolopyridine, phenelzine, tranylcypromine, desipramine, moclopemide, dothiepin, doxepin, paroxetine, oxazine, viloxazine, mirtazapine and nefazadone amongst others.

Particularly of interest is the combination of a SSRI with L-phenylalanine, l-tyramine or L-tryptophan. The case studies below demonstrate effectiveness of fluoxetine, lofepramine and citalapram in such a combination. The same effect is expected for other SSRIs, e.g. paroxetine.

A neurotransmitter inducer is a component which enhances or triggers production of a neurotransmitter.

A preferred neurotransmitter precursor for use in the present invention is L-phenylalanine (LPA). Another is L-tyramine.

Other amino acids such as L-tyrosine or other compounds such as tyramine may also find use in the present invention as a neurotransmitter, inducer or precursor. L-tryptophan is also useful, as indicated above.

Compounds may be provided as a metabolite of a precursor. For example, L-phenylalanine may be provided as a metabolite of aspartame.

If the combination for treatment includes vitamin B₁₂, this may be in the form of cyanocobalamin or hydroxycobalamin, to be administered orally or intramuscularly.

The compositions provided herein may comprise an antidepressant and a neurotransmitter precursor or inducer, or any other combination of components disclosed herein, as combined (simultaneous or sequential) actives. However, compounds may be employed which mimic a given active in improving diagnostic status and/or ameliorating one or more symptoms of depression (mimetics). Such compounds and their use are within the scope of the present invention. Also within the scope of the present invention are derivatives or analogues of the antidepressant which retain the antidepressant (e.g. MAOI, SSRI or SNRI) activity, respectively.

In accordance with the present invention, the compositions provided may be administered to individuals. Administration is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors. Dose regimens for the MAOIs, SSRI and tricyclic antidepressants may be within the range used for the treatment of depression (for which the standard starting dose of lofepramine is 140 mg per day). With the proviso that the prescribing physician will be able to decide suitable and safe dosage levels, a possible range for administration of antidepressants is 10-210 mg per day, although 50-70 mg per day may be suitable. For the neurotransmitter precursors or inducers, a range of 100 mg to 5 g per day, preferably 500-2000 mg/d (mg per day) may be employed, the dose increasing in proportion to the level of antidepressant or MAOI employed.

As an example, a 70 mg dose of lofepramine may be combined with 500 mg of L-phenylalanine given in the morning, this being supplemented with a further 500 mg of L-phenylalanine given in the afternoon.

Where vitamin B₁₂ is co-administered, the amounts may be those generally recommended for daily intake of the vitamin or may be greater than that recommended as average daily intake. The preferred average dosage range for vitamin B₁₂ in the invention is from 1 mg every 3 months up to 1 mg every 3 days. When symptoms are severe, this may be 1 mg intramuscular hydroxycobalamin per week in an 8-10 week course at the start of treatment, perhaps reduced to 1 mg every 10 days as treatment progresses. The desired dosage level of vitamin B₁₂ may conveniently be given by weekly intramuscular injection, but doses ranging from 5 μg to 10 mg may be given daily orally.

Pharmaceutical compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. L-tryptophan and L-phenylalanine are available in 500 mg tablets.

A combined oral preparation in single tablet form, containing all these components A, B and C or C′, or for example components B and C or C′, is feasible. Alternatively, a treatment pack may contain the components separately.

### EXAMPLES

Case #1

A 49 year old male with chronic endogenous depression who was depressed, sometimes relatively severely, for over 20 years was being treated with 30 mg fluoxetine once daily, requiring increasing doses to sustain antidepressant effects. Previous treatment with prothiaden had been relatively ineffective and the patient complained of side effects. Thus a return to tricyclic antidepressants was not recommended. He was commenced on a combination of fluoxetine 30 mg, L-phenylalanine 500 mg and vitamin B₁₂ 2000 μg orally, all once daily, with a sudden improvement in his depressive condition. His mood had improved and indeed he was as a consequence able to re-establish a relationship with his social partner that very day. He continues to improve clinically on the combination treatment.

Case #2

A woman of 42 years, with endogenous acute non-psychotic depression, had failed to respond to therapy with a number of conventional SSRI antidepressants. While she was taking citalapram (10 mg daily), the amino acid precursor L-tyramine (500 mg daily) was added to her regime with good effect on her depression. She was not taking vitamin B₁₂.

Case #3

A woman of 40 years, with moderate depression, both exogenous and endogenous, was treated with lofepramine (70 mg twice daily), vitamin B₁₂ (injection intramuscularly, 1 mg every two weeks) and L-phenylalamine (LPA), with rapid onset of symptom relief. She then stopped taking the lofepramine, while continuing the LPA and vitamin B₁₂. Her depression recurred, and she was started on fluoxetine (20 mg daily), continuing the previous dosages of LPA and vitamin B₁₂, with very rapid and effective onset of anti-depressant action. Then she discontinued fluoxetine and recommenced it three months later (while throughout continuing the LPA and vitamin B₁₂), with equally rapid and effective onset of anti-depressant action. Note that the usual reduction of effect, on stopping and restarting an antidepressant, was absent.

It will be apparent to those skilled in the art that variations and modifications to the specific embodiments disclosed herein may be made without departing from the scope of the invention.

1.-15. (canceled)

16. Method of treatment of a patient suffering from a form of depression, particularly endogenous depression, comprising administering to the patient any one of the following combinations of components:
I. A, B and C or C′
II. A and B
III. Band C or C′
IV. A and C or C′
wherein
A is an antidepressant,
B is vitamin B12, and
C is a precursor or inducer of a neurotransmitter (other than L-tryptophan),
C′ is L-tryptophan,
said components being administered simultaneously or separately, in amounts which in combination have the effect of ameliorating the depression.

17. Method according to claim 16 wherein the depression is chronic depression or acute (severe) non-psychotic depression.

18. Method according to claim 18, wherein A is an antidepressant exhibiting SSRI or SNRI activity.

19. Method according to claim 18, wherein A is lofepramine, fluoxetine, citalapram or paroxetine.

20. Method according to claim 16, wherein B is in the form of cyanocobalamin or hydroxycobalamin.

21. Method according to claim 16, wherein C is L-phenylalanine, L-tyrosine or tyramine.

22. Method according to claim 16, wherein the combination of components is L-tryptophan and a SSRI.

23. A pharmaceutical composition for treatment of depression having a combination of pharmaceutically active components consisting only of or including any one of the combinations set out in claim 16.
